(19) United States
(12) Patent Application Publication
(10) Pub. No.: US 2024/0245729 A1
(43) Pub. Date: Jul. 25, 2024

(54) METHODS FOR PRIMING ALLOGENEIC CULTURED KERATINOCYTE COMPOSITIONS FOR TOPICAL USE

(71) Applicant: Stratatech Corporation, Madison, WI (US)

(72) Inventors: Allen R. Comer, Madison, WI (US); Kenneth R. Gratz, Madison, WI (US)

(21) Appl. No.: 18/565,003

(22) PCT Filed: May 27, 2022
(86) PCT No.: PCT/US2022/031399

§ 371 Date: Nov. 28, 2023

Related U.S. Application Data
(60) Provisional application No. 63/194,687, filed on May 28, 2021.

(51) Int. Cl. A61K 35/36; A61K 9/00; A61K 35/33; A61K 38/39; C12N 5/071

(52) U.S. Cl. CPC A61K 35/36; A61K 9/0014; A61K 35/33; A61K 38/39; C12N 5/0698; C12N 2500/12; C12N 2500/20; C12N 2500/32; C12N 2500/34; C12N 2500/38; C12N 2533/54

(57) ABSTRACT

The present disclosure encompasses methods for priming an allogeneic cultured keratinocyte composition for topical use.

### FIELD OF THE DISCLOSURE

The present disclosure involves a method for priming an allogeneic cultured keratinocyte composition for topical use, particularly in adults with thermal burns. Therefore, the present disclosure generally relates to the field of medicine, in particular dermatology and burn treatments.

### BACKGROUND

Patients who suffer severe thermal burns face a long and painful recovery. When surgical intervention is required, such as in some deep partial-thickness burns, a patient's own skin or skin from a cadaver may be grafted to the wounded area. Although autografts reduce the chance of rejection that comes with use of cadaver skin, it requires the patient to endure further trauma. What is needed are grafts that promote healing and have a low chance of being rejected by a patient.

### BRIEF DESCRIPTION OF THE FIGURES

FIG. 1 depicts an outer carton and foil pouch packaging for a composition of the present disclosure.

FIG. 2 depicts a product dish containing a composition of the present disclosure.

FIG. 3 depicts a product dish and insert tray containing a composition of the present disclosure.

FIG. 4 depicts a bottle contain hold solution within a laminated foil pouch.

FIG. 5 depicts a hold dish contained within a clear pouch.

FIG. 6 depicts a hold dish handled in a sterile field.

FIG. 7 depicts a sterile operator (gray gloves) removing a sterile hold dish presented by the nonsterile operator (white gloves).

FIG. 8 depicts a sterile operator placing the hold dish in the sterile field (gray rectangle).

FIG. 9 depicts the removal of the foil pouch.

FIG. 10 depicts removing a closed product dish from the foil pouch.

FIG. 11 depicts the placement of the product dish in a nonsterile area.

FIG. 12 depicts a nonsterile operator aspetically pouring hold solution into the hold dish.

FIG. 13 depicts a nonsterile operator (white gloves) opening the product dish.

FIG. 14 depicts a sterile operator (gray gloves) removing the insert tray.

FIG. 15 depicts a sterile operator placing an insert tray into the hold dish.

FIG. 16 depicts meshing a composition of the present disclosure.

### SUMMARY OF THE DISCLOSURE

Among the various aspects of the invention is a method of priming an allogeneic cultured keratinocyte composition for topical use. The method includes removing the allogeneic cultured keratinocyte composition form sterile packaging and contacting the allogeneic cultured keratinocyte composition with a hold solution, wherein the hold solution includes a source of nutrients and osmotic regulators. The method may further include thawing the allogeneic cultured keratinocyte composition, meshing the allogeneic cultured keratinocyte composition, cutting or trimming the allogeneic cultured keratinocyte composition, and/or warming the hold solution to 35° C. to 39ºC. The contacting step may be at least 15 minutes and up to four hours.

The hold solution of the disclosed method may include 4-(2-hydroxyethyl)-1-piperazinethanesulfonic acid and/or an F-12 nutrient mixture. The cultured keratinocyte composition may also include an allogeneic cellularized scaffold, wherein the scaffold further comprises dermal fibroblasts and murine collagen. The hold solution and the allogeneic cultured keratinocyte composition may be clinically safe to a patient in need thereof. The hold solution and the allogeneic cultured keratinocyte composition may produce no immunological responses or adverse events to a patient in need of thermal burn treatment.

### DETAILED DESCRIPTION

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein “adverse events” refers to any untoward medical occurrence associated with the use of the hold solution or the allogeneic cultured keratinocyte composition. Adverse events may include immunological responses.

As used herein, “clinically safe” refers to a level of safety regarding use of a medical device/composition in which the potential benefits of using the device/composition outweigh the risks of using the device/composition.

As used herein, “immunological response” refers to the reaction of a patient's immune system in response to internal or external stimuli.

### I. Method

Disclosed herein are methods of priming an allogeneic cultured keratinocyte composition for topical use. The method may include removing the allogeneic cultured keratinocyte composition from sterile packaging and contacting the allogeneic cultured keratinocyte composition with a hold solution comprising a source of nutrients and osmotic regulators. In some aspects, the hold solution and the allogeneic cultured keratinocyte composition may be clinically safe to a patient in need thereof. In some additional aspects, the hold solution and the allogeneic cultured keratinocyte composition produce no immunological responses or adverse events to a patient in need of thermal burn treatment. In some additional aspects, the allogeneic cultured keratinocyte composition may be cryopreserved in a cryopreservation solution.

In some aspects, the contacting step may take at least about 15 minutes and up to about 5 hours. In some examples, the contacting step may take about 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours. In some additional examples, the contacting step may take about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, about 55 minutes to about 1 hour, about 1 hour to about 1.5 hours, about 1.5 hours to about 2 hours, about 2 hours to about 2.5 hours, about 2.5 hours to about 3 hours, about 3 hours to about 3.5 hours, or about 3.5 hours to about 4 hours. In some examples, the contacting step may be aseptically pouring the hold solution into a holding dish and then gently lowering the allogeneic cultured keratinocyte composition into the holding dish. The holding dish may be a sterile bowl, plate, tray, or other sterile surface.

In some embodiments, the allogeneic cultured keratinocyte composition may include an allogeneic cellularized scaffold, wherein the scaffold comprises dermal fibroblasts and murine collagen. The dermal fibroblasts may promote wound healing by generating connective tissue.

In some embodiments, the method may further comprise thawing the allogeneic cultured keratinocyte composition. The thawing step may be accomplished prior to the step of removing the allogeneic cultured keratinocyte composition from sterile packaging.

In some embodiments, the method may further comprise meshing the allogeneic cultured keratinocyte composition. Meshing may be accomplished with autograft meshing devices known in the art. The meshing devices may be crushing or noncrushing. The allogeneic cultured keratinocyte composition may be meshed at ratios of up to 1:1.

In some embodiments, the method may further comprise cutting or trimming the allogeneic cultured keratinocyte composition. The allogeneic cultured keratinocyte composition may be cut or trimmed in any manner at a physician's discretion to appropriately apply the allogeneic cultured keratinocyte composition to a patient. In some examples, the allogeneic cultured keratinocyte composition may be trimmed or cut to fit the shape and size of a patient's wound area.

In some embodiments, the method may further comprise warming the hold solution to about 35° C. to about 39ºC. In some examples, the hold solution may be warmed to about 35° C., 36° C., 37° C., 38ºC, or about 39ºC. In some additional examples, the hold solution may be warmed to about 34° C. to about 35° C., about 35° C. to about 36° C., about 36° C. to about 37° C., about 37° C. to about 38° C., or about 38° C. to about 39° C. Warming the hold solution may be accomplished using a warming oven, a water bath, or by using other devices known in the art.

In some exemplary embodiments, the disclosed method is practiced after a wound bed in a patient is prepared through excision and/or debridement. This allows physicians to determine the amount of allogeneic cultured keratinocyte compositions that will be required to adequately treat the wound.

### II. Hold Solution Composition

The disclosed method includes contacting the allogeneic cultured keratinocyte composition with a hold solution. When warmed, the hold solution provides an efficient medium for heat distribution. Additionally, when the allogeneic cultured keratinocyte composition is cryopreserved in a cryopreservation solution, the hold solution may dilute residual cryopreservation solution contained in the allogeneic cultured keratinocyte composition. The hold solution of the disclosed method includes a source of nutrients and osmotic regulators. The hold solution thus provides a healthy environment for the allogeneic cultured keratinocyte composition during preparation for use.

The hold solution includes a source of nutrients. In some embodiments, the source of nutrients may include Ham's F-12 nutrient mixture. Those with skill in the art will understand that Ham's F-12 nutrient mixture contains a variety of amino acids, vitamins, inorganic salts, and other components that are useful for serum-free growth of mammalian cells. In some embodiments, the hold solution may include DMEM, Ham's F-10, Medium 199, MEM, or RPMI.

In some embodiments, the hold solution may include a buffer. In some aspects, the buffer may include 4-(2-hydroxyethyl)-1-piperazineethanesulfonic acid (HEPES), 3-(N-morpholino)propanesulfonic acid, 2-(N-morpholino)ethanesulfonic acid, N, N-bis(2-hydroxyethyl)-2-aminoethanesulfonic acid, 3-morpholino-2-hydroxypropanesulfonic acid, N-(2-acetamido)-2-aminoethanesulfonic acid, (tris(hydroxymethyl)methylamino)propanesulfonic acid, bicine, or tricine. In one example, the buffer is HEPES. In some aspects, the buffer may provide a buffered system in the pH range of about 7.0 to about 7.4.

### EXAMPLES

### Example 1: Hold Solution

An example hold solution was prepared having the following composition:
(a) Glycine with a concentration of about 0.25 mM/L;
(b) L-alanine with a concentration of about 0.05 mM/L;
(c) L-arginine hydrochloride with a concentration of about 0.70 mM/L;
(d) L-asparagine-H₂O with a concentration of about 0.05 mM/L;
(e) L-Aspartic acid with a concentration of about 0.05 mM/L;
(f) L-cysteine hydrochloride-H₂O with a concentration of about 0.10 mM/L;
(g) L-cysteine 2HCI with a concentration of about 0.10 mM/L;
(h) L-glutamic acid with a concentration of about 0.05 mM/L;
(i) L-glutamine with a concentration of about 2.50 mM/L;
(j) L-histidine hydrochloride-H₂O with a concentration of about 0.15 mM/L;
(k) L-isoleucine with a concentration of about 0.42 mM/L;
(I) L-Leucine with a concentration of about 0.45 mM/L;
(m) L-lysine hydrochloride with a concentration of about 0.50 mM/L;
(n) L-methionine with a concentration of about 0.12 mM/L;
(o) L-phenylalanine with a concentration of about 0.22 mM/L;
(p) L-proline with a concentration of about 0.15 mM/L;
(q) L-serine with a concentration of about 0.25 mM/L;
(r) L-threonine with a concentration of about 0.45 mM/L;
(s) L-tryptophan with a concentration of about 0.04 mM/L;
(t) L-tyrosine disodium salt dihydrate with a concentration of about 0.21 mM/L;
(u) L-valine with a concentration of about 0.45 mM/L;
(v) Biotin with a concentration of about 0.00001 mM/L;
(w) Choline chloride with a concentration of about 0.06 mM/L;
(x) D-calcium pantothenate with a concentration of about 0.005 mM/L;
(y) Folic acid with a concentration of about 0.006 mM/L;
(z) Niacinamide with a concentration of about 0.02 mM/L;
(aa) Pyridoxine hydrochloride with a concentration of about 0.01 mM/L;
(bb) Riboflavin with a concentration of about 0.0006 mM/L;
(cc) Thiamine hydrochloride with a concentration of about 0.006 mM/L;
(dd) Vitamin B12 with a concentration of about 0.0005 mM/L;
(ee) i-Inositol with a concentration of about 0.07 mM/L;
(ff) Calcium chloride anhydrous with a concentration of about 1.05 mM/L;
(gg) Cupric sulfate with a concentration of about 0.000005 mM/L;
(hh) Ferric nitrate with a concentration of about 0.0001 mM/L;
(ii) Ferric sulfate with a concentration of about 0.002 mM/L;
(jj) Magnesium chloride anhydrous with a concentration of about 0.30 mM/L;
(kk) Magnesium sulfate anhydrous with a concentration of about 0.41 mM/L;
(II) Potassium chloride with a concentration of about 4.16 mM/L;
(mm) Sodium bicarbonate with a concentration of about 14.29 mM/L;
(nn) Sodium chloride with a concentration of about 120.61 mM/L;
(00) Sodium phosphate dibasic anhydrous with a concentration of about 120.61 mM/L;
(pp) Sodium phosphate monobasic with a concentration of about 0.45 mM/L;
(bb) Zinc sulfate with a concentration of about 0.002 mM/L;
(rr) D-glucose with a concentration of about 17.51 mM/L;
(ss) HEPES with a concentration of about 15.02 mM/L;
(tt) Hypoxanthine Na with a concentration of about 0.02 mM/L;
(uu) Linoleic acid with a concentration of about 0.0001 mM/L;
(vv) Lipoic acid with a concentration of about 0.0005 mM/L;
(ww) Putrescine 2HCI with a concentration of about 0.0005 mM/L;
(xx) Sodium pyruvate with a concentration of about 0.50 mM/L; and
(yy) Thymidine with a concentration of about 0.002 mM/L.

### Example 2: Clinical Trial Results

In a clinical trial that included 119 subjects (101 patients with deep partial-thickness thermal burns, 18 patients with full-thickness complex skin defects), the disclosed method of priming an allogeneic cultured keratinocyte composition was used. The results of the trial showed that the safety profile of the disclosed allogeneic cultured keratinocyte composition was similar to that of autograft sites. Specifically, the wound-related events including erythema, swelling, local warmth, and wound site infections were similar to that of autografting. In particular, no clinically significant immunological responses were observed when using the hold solution in conjunction with the allogeneic cultured keratinocyte composition were observed. Table 1 shows the adverse events that occurred during the clinical trial.

TABLE 1

| Adverse Reaction | Number of patients (%)* |
|---|---|
| Pruritus (itching) | 13 (11) |
| Blisters | 5 (4) |
| Hypertrophic Scar | 3 (3) |
| Impaired Healing | 3 (3) |

*If a patient had more than one incidence of an adverse reaction, the patient was counted only once.

This demonstrated that the hold solution and the allogeneic cultured keratinocyte composition were well-tolerated and safe to use. Importantly, there were no reports of rejection of the allogeneic cultured keratinocyte composition.

### Example 3: Preparation of the Composition

The allogeneic cultured keratinocyte composition (e.g. STRATAGRAFT) is an allogeneic cellularized scaffold product indicated for the treatment of adults with thermal burns containing intact dermal elements for which surgical intervention is clinically indicated (deep partial-thickness burns).

STRATAGRAFT is for topical application to a prepared wound bed (excision/debridement). A STRATAGRAFT construct is an approximately 100 cm2 (approximately 8 cm by 12.5 cm) off-white rectangle. A STRATAGRAFT construct may be trimmed to fit the shape and size of the wound area. The surface area of STRATAGRAFT to be applied should be equal to the surface area of the wound to be treated. Multiple constructs may be applied to cover large wound areas. If multiple constructs are required to cover the wound area, abut or adjoin the STRATAGRAFT constructs, and it is not necessary to overlap the edges. Each construct is for application to a single patient only. The Hold Solution is a cell-culture medium that is not supplemented with growth factors.

The allogeneic cultured keratinocyte composition was prepared by the following steps:

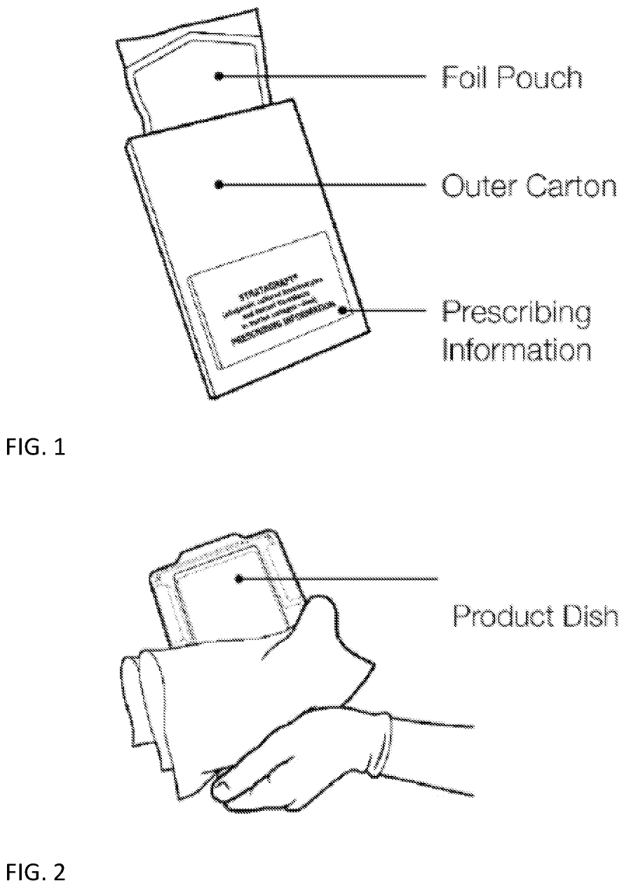

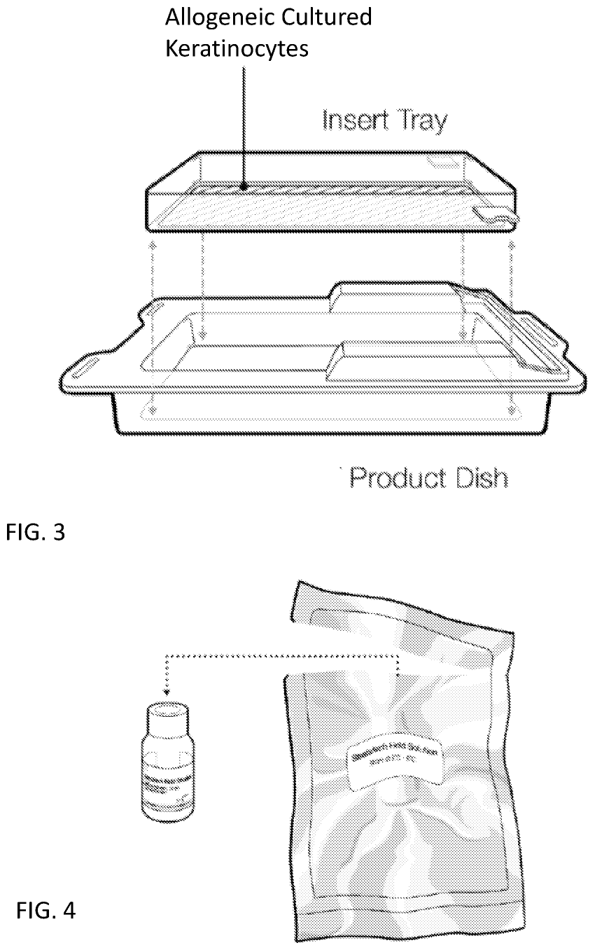

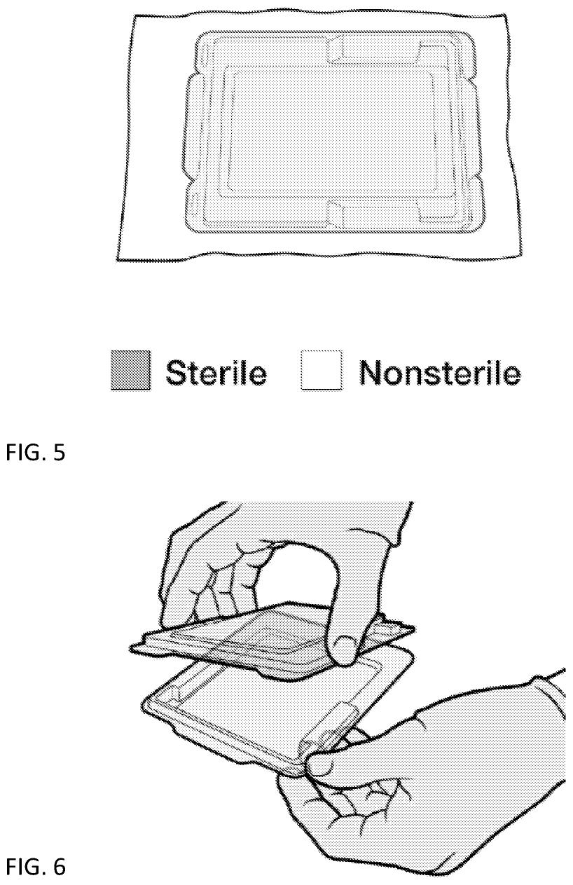

The allogeneic cultured keratinocyte composition was provided in a carton and enclosed in a foil pouch (FIG. 1). Within the foil pouch, the allogeneic cultured keratinocyte composition was contained in a polystyrene tray and loosely adhered to a polycarbonate membrane contained within the polystyrene tray (FIG. 2 and FIG. 3). The polystyrene tray was contained within a product dish (FIG. 2). The hold solution was provided in a plastic bottle contained in a laminated, foil pouch (FIG. 4). A holding dish was provided in a clear pouch (FIG. 5) consisting of a top portion and a bottom portion (FIG. 6).

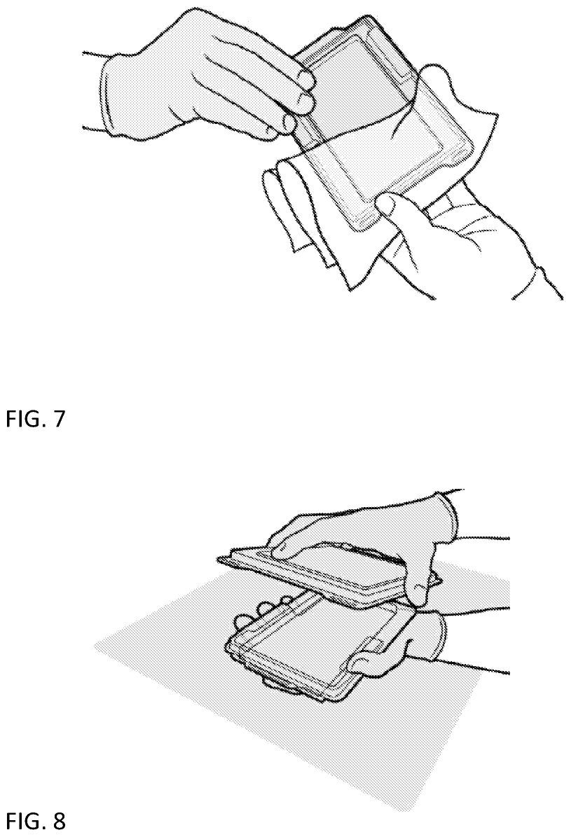

The hold solution was then removed from the laminated, foil pouch. The hold solution was then placed in a warming oven was used to warm the hold solution to 35-39ºC for at least 45 minutes prior to use, or in a water bath for at least 15 minutes prior to use. When the water bath was used, the cap or threads of the bottle were not submerged in the bath. An operator then peeled open the seal of the clear pouch containing the hold dish (FIG. 7), which was then removed aseptically from the pouch and placed in a sterile field by another operator (FIG. 8).

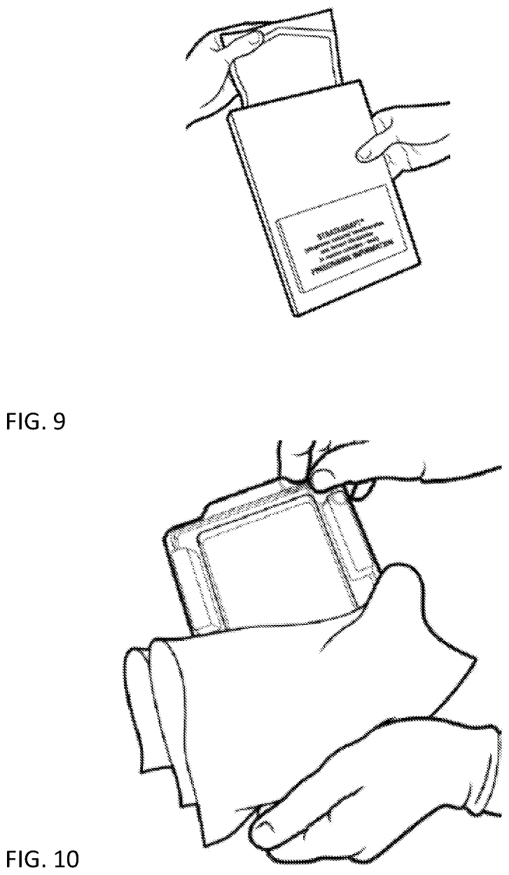

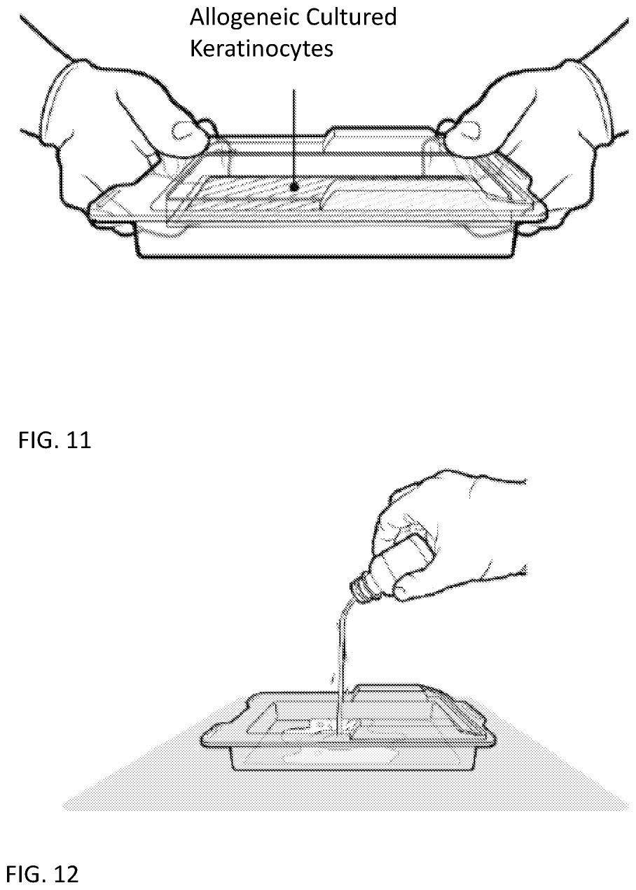

The allogeneic cultured keratinocyte composition was then removed from the carton (FIG. 9). The foil pouch was then peeled open (FIG. 10) and the polystyrene tray was removed and placed on a nonsterile surface (FIG. 11).

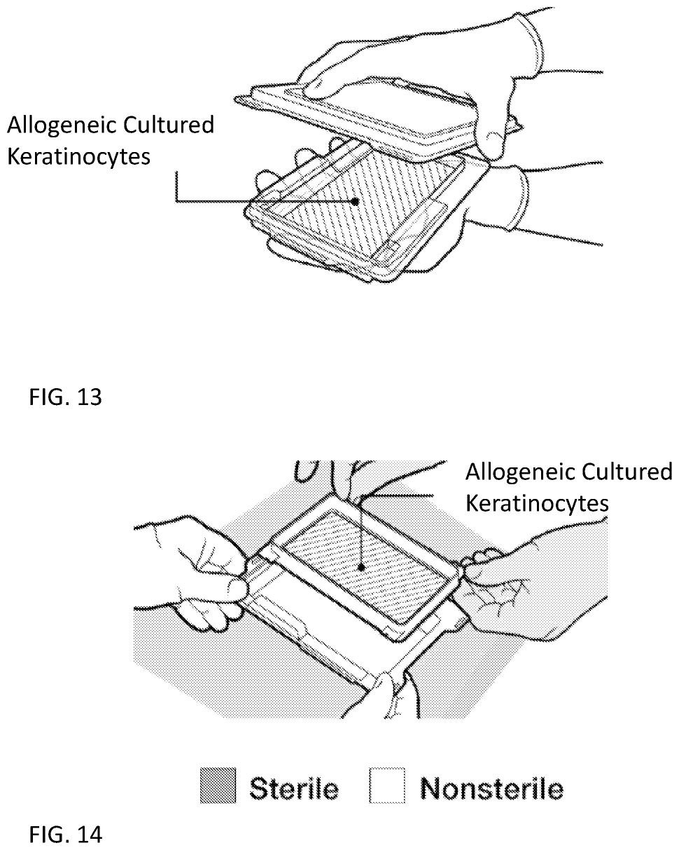

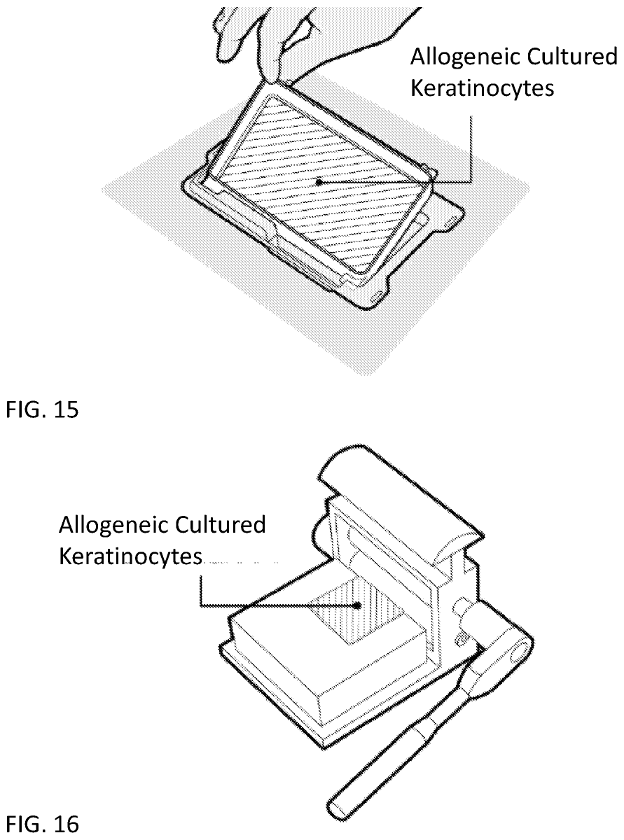

The hold solution was removed from the warming oven and immediately poured into the sterile hold dish using aseptic technique (FIG. 12). A nonsterile operator then removed the lid from the product dish without contacting the polystyrene tray (FIG. 13). A sterile operator then aseptically removed the polystyrene tray from the product dish using either sterile, gloved fingers or forceps (FIG. 14). The sterile operator then placed the polystyrene tray into the hold dish, beginning with one edge and lowering it to the opposite edge to minimize trapping bubbles beneath the insert tray (FIG. 15). If bubbles were trapped beneath the insert tray, the sterile operator gently lifted the insert tray and placed it slowly back down in the hold solution. The insert tray containing the allogeneic cultured keratinocyte composition was then maintained in the hold solution for at least 15 minutes, but no longer than 4 hours.

Last, the allogeneic cultured keratinocyte composition was removed from the polycarbonate membrane using sterile, gloved fingers or a pair of atraumatic forceps. The allogeneic cultured keratinocyte composition was then meshed up to a ratio of 1:1 (FIG. 16). The allogeneic cultured keratinocyte composition was not allowed to dry: the mesher and tissue board were moistened as needed using hold solution, sterile 0.9% normal saline, or lactated Ringer's solution to prevent drying.

What is claimed is:

1. A method of priming an allogeneic cultured keratinocyte composition for topical use, the method comprising:
removing the allogeneic cultured keratinocyte composition from sterile packaging; and
contacting the allogeneic cultured keratinocyte composition with a hold solution comprising a source of nutrients and osmotic regulators.

2. The method of claim 1, wherein the hold solution comprises 4-(2-hydroxyethyl)-1-piperazineethanesulfonic acid.

3. The method of claim 1, wherein the hold solution comprises an F-12 nutrient mixture.

4. The method of claim 1, further comprising thawing the allogeneic cultured keratinocyte composition.

5. The method of claim 1, further comprising meshing the allogeneic cultured keratinocyte composition.

6. The method of claim 1, further comprising cutting or trimming the allogeneic cultured keratinocyte composition.

7. The method of claim 1, wherein the allogeneic cultured keratinocyte composition comprises an allogeneic cellularized scaffold, wherein the scaffold further comprises dermal fibroblasts and murine collagen.

8. The method of claim 1, wherein the hold solution and the allogeneic cultured keratinocyte composition are clinically safe to a patient in need thereof.

9. The method of claim 1, wherein the hold solution and the allogeneic cultured keratinocyte composition produce no immunological responses or adverse events to a patient in need of thermal burn treatment.

10. The method of claim 1, further comprising warming the hold solution to 35 to 39° C.

11. The method of claim 1, wherein the contacting step is at least 15 minutes and up to 4 hours.
